(19) United States
(12) Patent Application Publication
(10) Pub. No.: US 2005/0239742 A1
(43) Pub. Date: Oct. 27, 2005

(54) Carrageenan-based formulations and associated methods of use

(75) Inventors: Virgil A. Place, Kawaihae, HI (US); Leland F. Wilson, Menlo Park, CA (US)

Correspondence Address: REED INTELLECTUAL PROPERTY LAW GROUP, 1400 PAGE MILL ROAD, PALO ALTO, CA, 94304-1124, US

(73) Assignee: VIVUS, Inc., Mountain View, CA (US)

(21) Appl. No.: 11/101,937

(22) Filed: Apr. 7, 2005

Related U.S. Application Data
(60) Provisional application No. 60/561,073, filed on Apr. 8, 2004.

(51) Int. Cl. A61K 31/737

(52) U.S. Cl. 514/54

(57) ABSTRACT

A method is provided for treating a female individual who suffers from or is prone to dyspareunia. The method involves vaginal and/or vulvar administration of a formulation comprising a therapeutically effective amount of a carrageenan and a pharmaceutically acceptable aqueous carrier. In addition, a carrageenan-based formulation is provided as a new composition of matter. Packaged kits for an individual to use in the administration of a carrageenan-based formulation as provided as well.

### CROSS-REFERENCE TO RELATED APPLICATIONS

This application claims priority under 35 U.S.C. §119(e)(1) to U.S. Provisional Patent Application Ser. No. 60/561,073, filed Apr. 8, 2004, the disclosure of which is incorporated by reference in its entirety.

### TECHNICAL FIELD

This invention relates generally to pharmaceutical formulations that contain carrageenan in a pharmaceutically acceptable aqueous carrier. Typically, such formulations exhibit an acidic pH level that matches the pH level of the local tissue environment to which the formulation is applied. The invention also pertains to the vaginal and/or vulvar administration of such formulations as a lubricant, particularly to a female individual who suffers from or is prone to dyspareunia.

### BACKGROUND

In a healthy female individual, lubricating fluid is usually present in the vagina. Upon sexual arousal, the adult female typically experiences an increase in vasocongestion and muscular tension, primarily in the genital region, through increased blood flow and elevated luminal oxygen tension. Increased vaginal surface lubrication may also occur as a result of plasma transudation that saturates the fluid resorptive capacity of the vaginal epithelium. The presence of additional lubricating fluid, in turn, provides enhanced comfort during sexual intercourse.

Conversely, the lack of sufficient vaginal lubrication causes vaginal tissue to become dry and irritated, which may result in painful sexual intercourse, and occasionally even bleeding. Insufficient vaginal lubrication may be caused by a number of factors. For example, decreased estrogen levels during menopause, after surgical removal of the ovaries, or after radiation therapy may contribute to vaginal dryness. Similarly, oral contraceptives and certain drugs such as antihistamines, antidepressants, blood pressure, and cardiac medications may impede vaginal fluid production. Furthermore, adverse psychological conditions, including stress, fatigue, and anxiety, may contribute to a decrease in vaginal fluid secretion. In some instances, a combination of hormonal and psychological factors may induce dryness temporarily after childbirth, particularly if the mother is breastfeeding. Administration of additional vaginal lubrication may enhance comfort during sexual intercourse, not only for female individuals who suffers from or is prone to dyspareunia, vaginal atrophy, vaginal itching and dryness, and vaginal pain, but also for those who report none of the above discussed symptoms.

A number of treatments to increase vaginal lubrication are commercially available. For example, vaginal lubricating suppositories are sold over-the-counter at most drug stores and supermarkets. Such suppositories, however, must first be inserted into the mid-vagina and then allowed to liquefy before adequate lubrication can be achieved. Such a delay between insertion and liquefaction tends to interfere with the pleasure and spontaneity of sexual intercourse. Fluid or gel vaginal lubricants are also commercially available, and include, for example, petroleum jelly, egg white, K-Y surgical lubrication jelly (hydroxyethyl-cellulose), Astroglide7, and Replens7. See, e.g., Semmens (1974) Medical Aspects of Human Sexuality 8:85-86, and Frishmen et al. (1992) Fertility and Sterility 58(3):630. Both fluid and gel lubricants suffer from well documented disadvantages, however. For example, K-Y Jelly is not ideal due to its suboptimal consistency, poor lasting quality, and lack of acceptable fragrance and taste. Oil-based lubricants such as petroleum jelly are contraindicated for use with a barrier contraceptive method (e.g., a male or female condom or diaphragm), because the latex rubbers used in the manufacture of these barrier contraceptives (for example, polyisoprene) are subject to degradation when exposed to oil-based lubricants, potentially lessening the reliability of the contraceptive device. In addition, lubricants containing glycerin are spermicidal and thereby impede sperm motility, even when only low concentrations of glycerin are present in the vagina. Accordingly, glycerin-containing personal lubricants are poorly suited for use if conception is a goal.

Carrageenan is a sulfated cell wall polysaccharide found in certain aquatic plants such as red algae, and contains repeating sulfated disaccharides of galactose and, sometimes, anhydrogalactose. Often employed in the commercial production of food, carrageenans have the ability to form gels at room temperature, with varying degrees of rigidity and/or melting points. Carrageenans may be classified into three different categories: kappa; iota; and lambda. These categories may be distinguished from one another according to their gelling properties and levels of protein reactivity. Kappa carrageenans produce strong rigid gels, exhibit some syneresis (i.e., contraction accompanied by the exudation of a liquid), and form helices with potassium ions. Introduction of calcium ions causes κ-carrageenans helices to aggregate. As a result, the gel contracts and becomes brittle. Kappa carrageenans are characterized by an ester sulfate content of approximately 25%. Iota carrageenans, on the other hand, form more flaccid, compliant, and elastic gels. In addition, iota carrageenans also form helices with calcium ions. Aggregation is limited in ι-carrageenans, and ι-carrageenans do not undergo syneresis. Iota carrageenans are characterized by an ester sulfate content of approximately 32%. Lambda carrageenans do not gel in water, exhibit a random distribution of polymer chains, and interact strongly with proteins. One commercial use for λ-carrageenan is to stabilize dairy products. Lambda carrageenans are characterized by an ester sulfate content of approximately 35%.

Carrageenan has been used as an ingredient in a number of formulations for vaginal administration. For example, International Patent Publication No. WO 00/56366 describes gel-microemulsion formulations that include carrageenan as a thickening agent. These formulations have been disclosed as spermicidal and antimicrobial compositions. Similarly, U.S. Pat. No. 6,017,521 to Robinson et al. describes a topical aqueous composition for treating bacterial vaginosis by lowering the vaginal pH to an acidic pH level, wherein carrageenan may be employed as an emulsifying agent. U.S. Pat. No. 5,069,906 to Cohen et al. describes the incorporation of the kappa and iota forms of carrageenan in a spermicidal gel formulation for use with an intravaginal contraceptive barrier such as a diaphragm. The patent states that, kappa and iota forms of carrageenan are used to help prevent fluid drainage from the vagina so as to enhance efficacy of the spermicide and minimize the risk of embarrassment and discomfort.

Recently, a number of researchers have proposed the use of a carrageenan gel for use as a vaginal microbicide. Sulfated polymers such as carrageenans have been reported to complex with the protein coating of viruses such as HIV, HTLV-1, and HSV, suggesting that carrageenan-based formulations may reduce the probability of transmission and infection. For example, Pearce-Pratt et al. (1996), “Sulfated Polysaccharides Inhibit Lymphocyte-to-Epithelial Transmission of Human Immunodeficiency Virus-1,” Biology of Reproduction, 54:173-182, describe an in vitro study suggesting that the iota type of carrageenan may be suitable for use as a vaginal microbicide. In addition, Maguire et al. (1998), “Carrageenan-Based Nonoxynol-9 Spermicides for Prevention of Sexually Transmitted Infections,” Sexually Transmitted Diseases 25(9):494-500, provide a comparison between carrageenan-based nonoxynol-9 formulations and existing over-the-counter nonoxynol-9-containing spermicides. The study concludes that the carrageenan-based spermicidal formulations are associated with a greater degree of protection against herpes simplex virus-2 than the over-the-counter spermicides. Furthermore, Coggins et al. (2000), “Preliminary Safety and Acceptability of a Carrageenan Gel for Possible Use as a Vaginal Microbicide,” Sex Transm. Inf. 76:480-483, describes a safety and acceptability study in which a gel formulation containing 2% λ-carrageenan in a Carbopol vehicle was evaluated by users. In some instances, subjects reported genital discomfort, itching, or burning. In addition, some subjects reported transient lower abdominal pain, urinary hesitancy, and/or feelings of a “heavy uterus.” Nearly one third of the subjects found the product “messy.”

Thus, there is a need for improved vaginal lubricant formulations that exhibit the demonstrated advantages associated with carrageenan as a component, but without the drawbacks of currently available products. In addition, opportunities exist in the art to enhance the performance and utility of carrageenan-based lubricant formulations. For example, carrageenan-based formulations may serve as lubrication for the instruments employed in routine pelvic or rectal examinations; or in more intimate situations, they may provide vaginal moisturization and/or coital lubrication.

### SUMMARY OF THE INVENTION

In one embodiment of the invention, a method is provided for treating a female individual who suffers from or is prone to dyspareunia. The method involves vaginal and/or vulvar administration of a formulation comprising a therapeutically effective amount of a carrageenan and a pharmaceutically acceptable aqueous carrier. Typically, dyspareunia is manifested by pain and discomfort during sexual intercourse, post-coital vaginal burning, vaginal dryness, vaginal itching, vaginal atrophy, pelvic aching, and/or urinary discomfort, and individuals who will benefit from the present method generally exhibit one or more of the foregoing symptoms. In some cases, dyspareunia is associated with female sexual dysfunction, generally an excitement stage sexual dysfunction. The carrageenan-based formulation used to treat dyspareunia is preferably substantially free of gelled materials and exhibits a viscosity in the range of approximately 1,000 to approximately 50,000 centipoise. Optimally, the formulation includes lambda carrageenan, and may or may not contain other carrageenans, i.e., iota carrageenan and kappa carrageenan. The formulation may also include one or more other inactive ingredients, i.e., conventional excipients used in topical and/or vaginal pharmaceutical compositions, and a preferred formulation includes a single additive or a combination of additives for maintaining formulation pH at an acidic level. In addition, the formulation may include a therapeutically effective amount of a pharmacologically active agent for treatment of a sexual dysfunction causing or otherwise associated with the dyspareunia, and/or for enhancing the patient's sexual desire and responsiveness.

In another embodiment, a carrageenan-based formulation is provided as a new composition of matter. The formulation consists essentially of approximately 1 wt. % to approximately 3 wt. % lambda carrageenan, at least one pH-adjusting additive for maintaining the pH of the formulation at an acidic level, and a pharmaceutically acceptable aqueous carrier suitable for vaginal, vulvar, or rectal administration. Generally, the formulation is substantially free of gelled materials, as alluded to above, and, accordingly, is substantially free of any carrageenans other than lambda carrageenan, i.e., substantially free of iota and kappa carrageenan. As indicated above with respect to formulations for the treatment of dyspareunia, the novel formulations exhibit a viscosity in the range of approximately 1,000 to approximately 50,000 centipoise, and may contain inactive excipients as well as a pharmacologically active agent, e.g., an agent for treating female sexual dysfunction and/or enhancing a female patient's sexual desire and responsiveness. The pH of the formulation is typically in the range of approximately 3.0 to approximately 5.0, and preferably in the range of approximately 3.75 to approximately 4.25. An optimal formulation pH is 4.0. The aforementioned formulation is useful for lubricating mucosal tissue, including vaginal tissue.

Also provided are packaged kits for an individual to use in the administration of a carrageenan-based formulation as provided herein, e.g., for the treatment of dyspareunia. Such kits are comprised of the inventive pharmaceutical formulations housed in a container during storage and prior to administration, and instructions for a patient to self-administer the formulation. The instructions may set forth directions for administering the formulation to the vagina and/or vulvar region of a female individual.

### DETAILED DESCRIPTION OF THE INVENTION

It is to be understood that unless otherwise indicated the invention is not limited with respect to specific formulation components, quantities thereof, methods of manufacture, or methods of use. It is also to be understood that the terminology used herein is for the purpose of describing particular embodiments only, and is not intended to be limiting.

It must be noted that, as used in this specification and the appended claims, the singular forms “a,” “an,” and “the” include plural referents unless the context clearly dictates otherwise. Thus, for example, reference to “a pharmacologically active agent” includes a mixture of two or more pharmacologically active agents; reference to “a buffer system” includes combinations of buffer systems; and the like.

In describing and claiming the present invention, the following terminology will be used in accordance with the definitions set forth below.

The terms “active agent,” “pharmacologically active agent,” and “drug” are used interchangeably herein to refer to a chemical compound that induces a desired pharmacological effect. The terms also encompass pharmaceutically acceptable, pharmacologically active derivatives of those active agents specifically mentioned herein, including, but not limited to, salts, esters, amides, prodrugs, active metabolites, analogs, and the like. When the terms “active agent,” “pharmacologically active agent,” and “drug” are used, then, it is to be understood that the terms encompass pharmaceutically acceptable, pharmacologically active salts, esters, amides, prodrugs, metabolites, and analogs of the active agent as well as the active agent per se.

By an “effective” amount or a “therapeutically effective amount” of a drug or pharmacologically active agent is meant a nontoxic but sufficient amount of the drug or agent to provide the desired effect. The amount that is “effective” will vary from subject to subject, depending on the age and general condition of the individual, the particular active agent or agents, and the like. Thus, it is not always possible to specify an exact “effective amount.” However, an appropriate “effective” amount in any individual case may be determined by one of ordinary skill in the art using routine experimentation.

By use of either term “female sexual dysfunction” or “sexual dysfunction” is meant any disorder or dysfunction that causes a decrease in or absence of female sexual responsiveness or female sexual desire. This includes any persistent or recurrent deficiency in the desire for sexual activity. It also encompasses decreases in the physiological response to sexual stimulation, for example, slowed or decreased erectile response of the female erectile tissues; slowed, decreased, or absent lubrication of the vagina; slowed, decreased, or absent ability to have orgasms; decreased intensity of or pleasure in orgasms; frigidity; sexual aversion; and disorders of female sexual desire and response that are secondary to a general medical condition such as the menopausal or post-menopausal state, radiotherapy of the pelvis, atherosclerosis, pelvic trauma or surgery, peripheral neuropathies, autonomic neuropathies, diabetes mellitus, and disorders of the innervation of any of the sexual organs. Substance-induced sexual dysfunction is also categorized among sexual response disorders that are secondary to a general medical condition, and include although not limited to, decreases in desire and responsiveness secondary to antidepressants, neuroleptics, antihypertensives, tobacco, opiates, alcohol, and any other drug found to decrease or eliminate any part of the sexual response cycle. Primary and secondary anorgasmia are included.

The terms “gel,” “gelled,” and “gelling” are used herein to refer to materials formed by the coagulation of a colloidal liquid. Often, gels have a fibrous matrix and fluid filled interstices. Unlike pure viscous fluids, gels are viscoelastic and can resist some mechanical stress without undergoing deformation.

The terms “lubricant,” “lubricating,” and “lubrication” are used herein to refer to any composition of matter that serves to reduce friction between an individual's tissue (typically vaginal tissue) and another solid object. Although any liquid (including water) may sometimes function as a “lubricant” in the broadest sense of the word, certain characteristics distinguish a preferred lubricant herein from water and other liquids that lack the general characteristics preferred for effective and comfortable lubrication. Characteristics of a preferred lubricant include all or some of the following: (1) slipperiness when rubbed against the surface of skin and/or mucosal tissue; (2) a relatively high viscosity as compared to water; (3) a relatively high affinity for skin or mucosal tissue, such that when it is spread smoothly and evenly across the contacted area upon application, it clings to the area of contact in a more substantial manner than water, which is easily wiped away; and, (4) a low volatility such that it does not evaporate quickly or become sticky.

It should be noted that as used herein, the term “lubricant” also encompasses “moisturizer” unless the context clearly indicates to the contrary. A vaginal or vulvar “moisturizer” is used to alleviate dryness, i.e., a lack of moisture in the vaginal and/or vulvar tissue, by promoting retention of moisture in the tissue. Similarly, the terms “lubricate” and “lubrication” encompass the terms “moisturize” and “moisturization,” respectively.

The terms “microbe” and “microbial” are used herein in their ordinary sense and refer to a microscopic living organism such as a bacterium, fungus, protozoa, or virus. Thus, the terms “antimicrobial” and “microbicide” are interchangeably used and refer to any agent capable of killing a microbe or rendering a microbe inactive.

The term “substantially,” as in, for example, “substantially free,” refers to the general absence of a substance and, more specifically, provides a maximum measure (e.g., quantity, volume, percentage, level, etc.) of a substance. For example, when a λ-carrageenan is “substantially free” from other forms of carrageenans, the other carrageenan forms represent no more than 25%, preferably no more than 10%, more preferably no more than 1%, and most preferably no more than 0.1% of the total carrageenan content in the formulation. Other uses of the term “substantially” involve an analogous definition.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, for example, “treating” sexual dysfunction, as the term is used herein, encompasses both prevention of sexual dysfunction in clinically asymptomatic individuals and treatment of dysfunction in a clinically symptomatic individual.

By the term “transdermal” drug delivery is meant delivery by passage of a drug through the skin or mucosal tissue and into the bloodstream.

The term “topical administration” is used in its conventional sense to mean delivery of a topical formulation that optionally contains drug or pharmacologically active agent to the skin or mucosa. Topical administration thus may include transmucosal administration.

Generally, “vaginal administration” of a pharmaceutical formulation involves administration to the distal several centimeters of the vagina. The terms “vulvar administration” are used herein to refer to application of a pharmaceutical formulation to the vulvar area of an individual. The term is intended to encompass application to the clitoris as well as to the surrounding vulvar area of an individual needing or desiring additional lubrication.

Thus, the invention relates generally to a method for providing treating a female individual who suffers from or is prone to dyspareunia. The method involves administering to the vagina and/or vulvar region of the individual a pharmaceutical formulation that comprises a therapeutically effective amount of a carrageenan and a pharmaceutically acceptable aqueous carrier. Typically the formulation contains λ-carrageenan without one or more other forms of carrageenan. Preferably, the formulation consists essentially of λ-carrageenan. In some instances, the formulation may include a means for maintaining the formulation pH at an acidic level. The method is particularly suited for individuals who exhibit or are prone to experiencing at least one symptom selected from pain and discomfort during sexual intercourse, post-coital vaginal burning, vaginal dryness, vaginal itching, vaginal atrophy, pelvic aching, and urinary discomfort. Further, the individuals may suffer from a sexual dysfunction, e.g., an excitement stage sexual dysfunction, manifested in part by dyspareunia. However, the invention also relates to a method for providing enhanced vaginal lubrication to a female individual, healthy or otherwise, who needs or desires additional lubrication.

Since the formulation is generally intended for topical administration to highly sensitive tissue such as vaginal and/or vulvar tissue, the texture and feel of the formulation are vitally important considerations. In order to provide optimum lubrication capability, the formulation should have a comfortable “slick” feel that approximates the touch and consistency of natural vaginal lubricant. It is well known that the viscosity of vaginal fluids changes throughout an individual's menstrual cycle. For most of the cycle, the vaginal fluids are thick and sticky in consistency. As ovulation approaches, however, the vaginal fluids often change in character and become clear, elastic, and gooey, similar to raw egg whites. In addition, the viscosity of vaginal fluids tends to decrease when vaginal ejaculation occurs as a result of excitation and/or orgasm. Preferably, the viscosity of the inventive formulation is similar to vaginal fluids present as a result of excitation and/or orgasm.

It has been found that among the three different types of carrageenans, λ-carrageenan exhibits superior lubricating capabilities (over the kappa and iota forms of carrageenan) due to its resistance to gelling. Thus, the formulation of the invention may be free or substantially free from forms of carrageenan other than λ-carrageenan. Preferably, at least 95 wt. % of the carrageenan in the formulation is typically λ-carrageenan. More preferably, at least 99 wt. % of the carrageenan in the formation is λ-carrageenan. Optimally, at least 99.9 wt. % of the carrageenan in the formation is λ-carrageenan. In the absence of substantial amounts of kappa- or iota-type carrageenans, the inventive formulation may be substantially free of gelled materials. In addition, such a formulation exhibits sustained lubricating effects without becoming sticky or gritty. However, as ι-carrageenan forms more flaccid, compliant, and elastic gels than κ-carrageenan, the formulation tolerates and therefore may contain more ι-carrageenan than κ-carrageenan. Thus, in some cases, the formulation may contain a small amount of ι-carrageenan but no κ-carrageenan.

Thus, the formulation may contain a dilute solution of λ-carrageenan, wherein the λ-carrageenan represents approximately 1.0 wt. % to approximately 3.0 wt. % of the formulation. In some instances, λ-carrageenan is present at a concentration in the range of approximately 1.5 wt. % to approximately 2.0 wt. %. Preferably, the formulation contains approximately 1.9 wt. % to approximately 2.1 wt. % λ-carrageenan. At lower concentrations, the formulation tends to be too runny. At higher concentrations, the formulation becomes too sticky. The formulation typically has a viscosity of approximately 1000 to approximately 50,000 centipoise, and preferably, the viscosity is approximately 10,000 to approximately 40,000 centipoise. Optimally, the viscosity is approximately 25,000 to approximately 40,000 centipoise.

Another feature of the inventive formulation is its pH. In general, the pH is maintained at a level appropriate with respect to the region of intended application. As alluded to above, the pH of healthy vaginal fluid is acidic. It is known that a number of commercially available lubricants are not pH-balanced. As a result of their repeated use and the accompanying change in vaginal pH, many commercially available personal lubricants inhibit the growth of healthy vaginal flora, resulting in repeated vaginal infections and disorders (thrush, cystitis, itching, and irritation). Thus, it is preferred that the administration of the formulation does not alter the pH of the fluids in the region of lubricant application. The non-bacteriostatic requirement is vitally important, because vaginal dryness is often associated with difficulties in maintaining healthy vaginal flora.

Accordingly, the inventive formulation also includes a means for maintaining the formulation at an acidic pH level, which typically involves the introduction of acid. Although the formulation becomes more acidic upon further introduction of acid, the formulation may be prepared such that it is not noticeably irritating to the vaginal mucosa with which it comes into contact. Thus, any nontoxic acid compatible with the tissue in the region of formulation administration may be used to reduce the pH of the formulation to below 7.0. Typically, a dilute acid is used. Suitable acids include, for example, acetic acid, ascorbic acid, benzoic acid, citric acid, hydrochloric acid, lactic acid, phosphoric acid, and salicylic acid. Larger organic acids may be used as well. For example, U.S. Pat. No. 6,017,521 to Robinson et al. describes the use of carboxylic acid polymers in an intravaginal formulation. Such polymers are described as containing a plurality of monomers of which at least approximately 80 percent contain at least one carboxyl functionality [—COOH] and a cross-linking agent present in a quantity sufficient to make the polymer water-swellable yet water-insoluble. As such, polymeric acids may increase the overall viscosity as well as the gel content of the formulation and may be appropriate in limited situations.

The preferred means for maintaining the formulation pH at an acidic level is a buffer system. Buffer systems are typically comprised of a weak acid and the salt of its anion, and are used to stabilize formulations against pH changes. Such buffer systems may be selected to stabilize the pH of the formulation at or near the region of tissue of intended formulation application. As the healthy vaginal pH is typically acidic, the buffer system is selected to maintain the formulation at a pH in the range of approximately 3.0 to approximately 5.0, preferably at a pH in the range of approximately 3.75 to approximately 4.25, and optimally at a pH of approximately 4.0. Buffer systems, which are commercially available from a number of sources (such as Sigma-Aldrich Co., St. Louis, Mo.), are well known in the art and may serve to stabilize the inventive formulation to these preferred pH ranges. “Good” buffers may be suitable for use with the invention at the higher pH ranges. Exemplary “good” buffer systems suitable for use include the following (as abbreviated by their common known acronyms): MES, Bis-Tris, ADA, ACES, IPES MOPSO, Bis-Tris Propane, BES, MOPS, HEPES, and TES. Similarly, phosphate buffer may be employed for pH levels of approximately 5.8 to approximately 7 as well as approximately 1.2 to approximately 3.1. Buffer systems based on benzoate, propionate, and acetates are available to maintain a pH of approximately 4.0.

The acidity of the formulation may provide additional benefits. For example, bacterial vaginosis clinically presents as a superficial vaginal infection with few irritating symptoms and no inflammatory response. Some noticeable symptoms, however, include an unpleasant smell, an elevated vaginal pH, a thin homogeneous discharge, the presence of Gardnerella clue cells, and a high succinate/lactate ratio. The increased pH level is thought to allow the anaerobes to grow and produce the amines that are present in a bacterial vaginosis infection. The amine odor produced in the vagina during an episode of vaginosis is known to become stronger at higher pH levels, due to the presence of unprotonated, volatile amines under basic conditions. The acidity of the formulation, however, tends to inhibit and/or combat the anaerobic infection. Therefore, using the inventive formulation, lubricants that have a pH of approximately 4 or less (e.g., pH 3-4, or more preferably pH 3.25-3.75) are favored for individuals suffering from vaginosis. Thus, for example, the formulation may employ an acetate buffer comprised of an acetate salt (e.g., sodium acetate) and acetic acid in order to maintain a pH level of approximately 4.

As alluded to above, an acidic vaginal pH is normally maintained by the lactobacilli. The acidity inhibits the growth of common yeasts, fungi, and other microbes that cause vaginal infections, because such microbes do not grow well at a pH of 5 or lower. When the quantity of vaginal lactobacilli is decreased, vaginal pH is increased. As a result, the risk of yeast and fungal growth, as well as of bacterial infections, in the vagina is increased. Thus, an effective-pH-adjusting amount of lactobacillus may be provided with the inventive formulation. The introduction of additional lactobacilli tends to promote healthy vaginal flora as well as decrease the pH of the formulation. In some instances, the pH maintaining means may include any combination of acids, buffers systems and lactobacilli.

It should be noted that carrageenans may not be completely stable at a pH level of less than about 5. For formulations having a relatively high acid pH level, e.g., 5-6.5, the pH maintaining means may be provided intimate contact with carrageenan without noticeable degradation in the stability of the formulation. When a lower pH level is desired, control release techniques may be used to ensure that the formulation exhibits an appropriate pH upon application. For instance, when an acid/salt buffer system is used, the acid may be provided in dispersed but segregated form such that it does not directly contact the carrageenan in the formulation until the formulation is applied. This may be achieved, for example, through the use of encapsulating microspheres that release the acid upon application of the formulation. Other techniques and materials that can be used to segregate acid from carrageenan are known in the art. Thus, the pH maintaining means is typically provided such that formulation exhibits an appropriate pH immediately prior to, shortly after and/or upon application of the formulation to the region of intended use, irrespective of the pH of the formulation during storage. For example, an acid may be added to the formulation before no more than about 1 day before administration of the formulation. Preferably, an acid is added to the formulation before no more than about 1 hour before administration of the formulation. The acid should be mixed thoroughly with the formulation to ensure that the formulation's pH is uniform throughout. This may be performed by either by hand or through the use of mixing devices known in the art or to be developed.

In its simplest form, no additional ingredient is provided with the inventive formulation. That is, the invention provides a formulation consisting of an aqueous solution of approximately 1.0 wt. % to approximately 3.0 wt. % λ-carrageenan and a means for maintaining the formulation pH at an acidic level. Such a formulation may serve as a general purpose lubricant suitable for topical administration to the vaginal and/or vulvar region of a female. The formulation may also be administered to the rectal and/or anal regions of a female or male. Thus, it is preferred that such a formulation be sterile. Such a formulation exhibits the antimicrobial properties associated with a carrageenan-based lubricant, as well as the optimized feel and texture associated with personal lubricants without gels. In addition, since carrageenan does not function as a spermicide, this particular formulation (or any formulation without a spermicide) is particularly suited for use in procreative applications. In addition, it should be noted that recent studies suggest that spermicides such as Nonoxynol-9 may cause epithelial disruption and/or disruption of the genital or anal mucosa, thereby increasing the risk of HIV transmission. Thus, this particular spermicide-free carrageenan-based formulation is particularly suited for reducing the risk of HIV transmission through rectal mucosa.

Depending on the intended use of the formulation, optional additional ingredients such as active agents, may be present. For example, the inventive formulation may be used in conjunction with active agents to treat female sexual dysfunction. A number of such active agents are described in U.S. patent application Ser. No. 09/929,818, entitled “Treatment of Female Sexual Dysfunction with Vasoactive Agents, Particularly Vasoactive Intestinal Polypeptide and Agonists Thereof,” inventors Place, Wilson, Doherty, Hanamoto, Spivack, Gesundheit, and Bennett, filed Aug. 13, 2001. In this application, a method is described for treating sexual dysfunction in a female individual comprising administering to the vagina and/or vulvar area a pharmaceutical formulation containing a selected vasoactive agent.

When a vasoactive agent is used with the inventive formulation, the vasoactive agent is preferably a vasodilator, with preferred vasodilators selected from the group consisting of prostaglandins, endothelin-derived relaxation factors, smooth muscle relaxants, leukotriene inhibitors, pharmaceutically acceptable salts, esters, analogs, derivatives, prodrugs, active metabolites, and inclusion complexes thereof, and combinations of any of the foregoing. The prostaglandin may be selected from the group consisting of naturally occurring prostaglandins, synthetic prostaglandins, pharmaceutically acceptable salts, esters, analogs, derivatives, prodrugs, active metabolites, and inclusion complexes thereof, and combinations of any of the foregoing. For example, the prostaglandin may be selected from the group consisting of PGE₀, PGE₁, PGA₁, PGB₁, PGF_{1α}, 19-hydroxy-PGA₁, 19-hydroxy-PGB₁, PGE₂, PGA₂, PGB₂, 19-hydroxy-PGA₂, 19-hydroxy-PGB₂, PGE₃, PGF_{3α}, PGI₂, and hydrolyzable esters thereof. Prostaglandin E₁ is particularly preferred. However, carboprost tromethamine, dinoprost tromethamine, gemeprost, metenoprost, sulprostone and tiaprost may be used as well.

Other active agents may be used as well. For example, the pharmacologically active agent may be selected from the group consisting of rho kinase inhibitors, melanocortin peptides, endothelin antagonists, growth factors and other peptidyl drugs, selective androgen receptor modulators (SARMs), neuropeptides, amino acids, serotonin agonists, serotonin antagonists, calcium channel blockers, potassium channel openers, potassium channel blockers, dopamine agonists, dopamine antagonists, non-androgenic steroid hormones, phosphodiesterase inhibitors, and combinations thereof.

As an alternative or additional option, spermicides may be included in the inventive formulation. Generally, any spermicide may be incorporated into the inventive formulation. For example, any one or combinations of nonoxynol-9, nonoxynol-15, octoxynol-9, and menfegol may be incorporated into the inventive formulation. Certain other surfactants may serve as spermicides as well. Nonoxynol-9 is a particularly preferred spermicide due to its proven efficacy and widespread use in conjunction with various over-the-counter contraceptive products. Since barrier devices such as cervical caps, diaphragms, and male and female condoms are often used in conjunction with spermicidal formulations, the inventive formulation typically excludes any compound that compromises the microbial barrier properties of an elastomeric and/or rubber-based material. For example, condoms other than those made from animal tissue are typically formed from elastic materials such as latex, e.g., polyisopropene or polyurethane. Thus, the inventive formulation preferably excludes petroleum products such as petroleum jelly and mineral oil, which are known to compromise the mechanical integrity as well as the microbial barrier properties of latex.

While carrageenan has been reported to exhibit antimicrobial activity with respect to protein-coated viruses such as HIV, it should be noted that the inventive formulation may contain an additional microbicide that inhibits viral, bacterial, fungal, and/or other infections. Typically, drugs such as acyclovir, clotrimazole, metronidazole, miconazole, nystatin, sulfas, and ticonazole may be incorporated within the formulation. Specifically suitable drugs that may be used include sulfabenzamide, sulfacetamide, sulfacytine, sulfatriazole and the like. Monoclonal antibodies such as those useful against cell surface components, or against pathogenic organisms such as HIV, may be incorporated into the inventive formulation as well. Furthermore, U.S. Pat. No. 5,980,477 to Kelly describes the use of zinc salts in conjunction with vaginal lubricants. According to this patent, the inclusion of water soluble, organic zinc salts of relatively low molecular weights (including zinc acetate, butyrate, gluconate, glycerate, glycolate, lactate, propionate, etc.) or highly ionizing inorganic salts, such as zinc chloride or sulfate, can also be used in formulations to reduce the risk of contracting sexually transmitted diseases such as AIDS or genital herpes. In addition, International Patent Publication No. WO 00/56366 describes the use of various antimicrobial AZT derivatives in pharmaceutical formulations. Other microbicides known in the art or that will be developed may be used as well.

It should be noted that mucosal tissue is generally more permeable than skin, and that transdermal delivery considerations must be accounted for when an active agent is employed. In some instances, permeation enhancers or permeation inhibitors may be incorporated into the inventive formulation.

In some instances, additional ingredients that are substantially pharmacologically inactive may be included as well. In general, these additional ingredients are physiologically acceptable and may be naturally occurring or may be of synthetic origin. That is, these ingredients may be gradually broken down into innocuous substances in the body (in cases in which they are absorbed by tissue to a significant degree through the skin or mucous membranes), or they are of a nature that allows them to be secreted by the vagina and washed cleanly from the skin. In either case, they do not foul or clog the pores in skin or mucous membranes, leave any unacceptable residues, or cause other adverse effects if used repeatedly over a span of months, during numerous acts of intercourse.

Thus, for example, additional ingredients may be added such as fragrances, coloring agents, and soothing or anti-swelling agents (such as lanolin, aloe vera extract, or hydrocortisone). Similarly, preservatives can be added. Typical preservatives known for use with feminine hygiene products include alcohol, ascorbyl palmitate, benzoic acid, butylated hydroxyanisole, butylated hydroxytoluene, chlorobutanol, ethylenediamine, ethylparaben, methylparaben, monothioglycerol, phenol, phenylethyl alcohol, propylparaben, sodium benzoate, sodium formaldehyde sulfoxylate, sodium metabisulfite, sorbic acid, sulfur dioxide, maleic acid, and propyl gallate. As these preservatives vary in the extent to which they are irritating to vaginal and/or vulvar tissue, the less irritating preservatives are preferred over those that are more irritating. Similarly, flavorings may be included as well, subject to the same general considerations as other additional ingredients.

In addition, a supplemental thickening agent may be included, such as acacia, agar, alginate, gum tragacanth, xanthan gum, collagen, carboxypolymethylene, polyvinylpyrrolidone, and polyacrylamide. Derivatives of cellulose that have been chemically treated to make them more hydrophilic (such as hydroxyethyl and hydroxymethyl derivatives, which have numerous additional hydroxy groups bonded to the starting cellulose molecules) may be used as well.

Furthermore, supplemental lubricating agents may be incorporated into the inventive formulation as well. Exemplary supplemental lubricating agents include glycerin (also called glycerine, glycerol, 1,2,3-propanetriol, and trihydroxypropane) and certain types of polyethylene glycol (PEG), such as PEG 200 or PEG 400 (the numbers indicate different molecular weight averages). Various other polymers (such as polypropylene glycol, polyisobutene, and polyethylene oxide) and certain naturally-occurring compounds (such as behenic acid, derived from various types of seeds and animal fats) and their derivatives (such as behenyl alcohol) may be used as well, since such polymers and compounds are sometimes used as lubricants in cosmetics and other formulations that contact the skin. In some instances, sugars and sugar-alcohols such as sorbitol, mannitol, and lactose, and some silicon compounds such as polydimethylsiloxane, may be used. Preferred supplemental lubricating agents include glycerin, propylene glycol, polyethylene glycol, and polypropylene glycol, due to their demonstrated biocompatibility, ease of synthesis, and widespread commercial availability.

In some instances, the inventive formulation may serve as a base for a pharmacologically active ointment, cream, emulsion, lotion, gel, solid, solution, suspension, foam, or liposomal formulation. Ointments that are semisolid preparations may contain petrolatum or other petroleum derivatives. Lotions are preparations that may be applied without friction, and are typically liquid or semiliquid preparations in which solid particles, including the active agent, are present in a water or alcohol base. Pharmaceutical emulsion formulations are generally formed from a dispersed phase (e.g., a pharmacologically active agent), a dispersion medium, and an emulsifying agent. Liposomes are microscopic vesicles having a lipid wall comprising a lipid bilayer, and can be used in conjunction with the inventive formulation and with an active agent as well. Additional information relating to the preparation and use of such pharmacologically active preparations is provided in U.S. patent application Ser. No. 09/929,818, entitled “Treatment of Female Sexual Dysfunction with Vasoactive Agents, Particularly Vasoactive Intestinal Polypeptide and Agonists Thereof,” inventors Place, Wilson, Doherty, Hanamoto, Spivack, Gesundheit, and Bennett, filed Aug. 13, 2001.

It should be noted, though, that a supplemental agent should be included only when it does not interfere with the enhanced properties of the inventive formulation. For example, supplemental agents that cause excessive gelling should be avoided when it is desirable to provide a substantially gel-free formulation. Similarly, when an active agent is included in the formulation, the supplemental agents should not adversely affect any active agent or other components of the formulation. In some instances, the certain supplemental agents may be provided only in a small amount.

The formulation is preferably, although not necessarily, administered on an as-needed basis. By “as-needed” dosing (also referred to as “pro re nata” dosing, “prn” dosing, and “on-demand” dosing or administration) is meant the administration of the formulation at a time just prior to that at which the presence of the formulation is wanted. For example, the formulation may be administered immediately prior to sexual activity to provide additional lubrication. In addition, the formulation may be employed when an individual desires or requires intimate moisturization. In some instance, vaginal dryness may be a chronic or semi-chronic condition, e.g., triggered by menopause, pregnancy, childbirth, nursing, or hysterectomy, requiring periodic treatment. In some instances, specific events may temporarily cause vaginal dryness. For example, use of any of the following may result in vaginal dryness: diaphragms; condoms; some hormonal contraceptives, fertility drugs; tampons; drying soaps; douches; antihistamines; decongestants; antibiotics; ulcer medication; high blood pressure medication; compounds associated with chemotherapy; antidepressants; tranquilizers; alcohol; narcotics; and sedatives. Other exemplary events that may cause vaginal dryness include, excessive exercise, extreme weight loss, stress, fatigue, and menstrual cycle changes. Administration of the inventive formulation may vary depending on the cause or systems associated with vaginal dryness.

It should be noted that when an active agent is included with the inventive formulation, pharmacokinetic considerations should be taken into account. For example, when the inventive formulation is used in conjunction with active agents to treat dyspareunia, as-needed administration may involve application of the formulation prior to sexual activity, e.g., approximately 0.25 to 72 hours, preferably approximately 0.5 to 48 hours, more preferably approximately 1 to 24 hours, most preferably approximately 1 to 12 hours, and optimally approximately 1 to 4 hours prior to anticipated sexual activity. In such as cases, priming doses or chronic administration, where “chronic” refers to drug administration at regular time intervals on an ongoing basis, are not needed. Thus, as-needed administration of the inventive formulation with an active agent may or may not involve administration of a sustained release formulation in advance of anticipated sexual activity, with drug release taking place throughout an extended drug delivery period typically in the range of approximately 4 to 72 hours. As will be appreciated by those in the fields of pharmacology and drug delivery, the upper ends of the aforementioned ranges will depend on the pharmacokinetics of the particular active agent administered.

In another embodiment, a packaged kit is provided that contains the pharmaceutical formulation to be administered, i.e., a pharmaceutical formulation for use in enhancing personal lubrication, and a container, preferably sealed, for housing the formulation during storage and prior to use. Typically, instructions for administering the formulation to enhance lubrication are provided as well. The instructions may be provided as written instructions on a package insert, a label, and/or on other components of the kit.

Depending on the type of formulation and the intended mode of administration, the kit may also include a device for administering the formulation (e.g., a transdermal delivery device). The administration device may be a dropper, a swab, a stick, or the tip of a pump or syringe. The formulation may be any suitable formulation as described herein. For example, the formulation may be provided in a gel or ointment contained within a tube. The kit may contain multiple formulations of different dosages of the same agent. The kit may also contain multiple formulations of different active agents.

The present kits will also typically include means for packaging the individual kit components, i.e., the pharmaceutical dosage forms, the administration device (if included), and the written instructions for use. Such packaging means may take the form of a cardboard or paper box, a plastic or foil pouch, etc. Packaging for the formulation is generally not critical to this invention, and there are a number of ways in which the invention may be packaged. For example, the inventive formulation may be packaged as a “stand-alone” lubricant, which is contained, shipped, and handled in a package that renders it convenient and useful as a lubricant during intercourse.

Thus, in some embodiments, the inventive formulation is packaged in a watertight tube made of deformable metal. Such tubes are typically sealed at one end by means such as crimping, and have an outlet orifice at an opposed second end, which can be covered and sealed by a removable and/or detachable member such as a threaded or flip-top cap. Such metallic foil tubes are commonly used to hold toothpaste, ointments, and gels (such as K-Y Lubricating Jelly and contraceptive gels). When squeezed to dispense a quantity of lubricant, the metallic tube may undergo plastic deformation and will not typically regain its original shape after the squeezing pressure is released. By avoiding the creation of a vacuum inside the tube, oxidative discoloration or degradation of the formulation is minimized in the tube.

Alternatively, a watertight tube with deformable plastic walls may be used. Such plastic tubes may be permanently sealed at one end (such as by heat-crimping), and have a removable cap covering an outlet orifice at the other end. Such tubes are commonly used to hold toothpaste, ointments, and gels (such as K-Y Lubricating Jelly and contraceptive gels). The cap can be a threaded screw-on cap, or a hinged flip-type cap that can be opened without detaching it from the tube, so that it cannot be lost, and that can be opened or closed easily with one hand. Between the two ends of the tube, the container has at least one deformable plastic wall, which in a preferred embodiment is essentially tubular, comparable to a toothpaste tube, with a transitional shoulder or neck region leading to the outlet orifice.

In some instances, a small watertight packet may contain a sufficient quantity (such as approximately 1 to 20 mL) of the inventive formulation for a single use during intercourse. Such packets can be made of plastic, metal foil, laminates, metallized plastic, or other suitable material. In some instances, the packet may be prenotched for easy opening. This type of small sealed packet allows the lubricant to be conveniently and discretely carried in a purse, pocket, glove compartment of a car, or other location without the large bulk or conspicuousness of a full-sized tube. As the packet may be disposed of after use, there is no need to provide a means to reseal the packet.

Similarly, a small single-dose container made of a breakable plastic or other material, which can be opened by breaking off a component that protrudes outward from the container, thereby unsealing an outlet orifice. This type of device is comparable to a miniature version of the plastic bottles with break-off tops that are widely used for non-carbonated children's drinks.

Furthermore, the formulation may be contained in a stiff-walled bottle. Such a bottle may be provided in an upright configuration, with a wall (typically cylindrical or with an elliptical or similar cross-sectional shape) made of plastic, glass, or other suitable material. When such containers have deformable plastic walls, they are simply another form of watertight tube, which can be squeezed when the cap is open to dispense the fluid contained therein.

Because lubricants are necessarily slippery, it may be difficult to open containers containing lubricants when the lubricant is present on an exterior surface of the container. This may occur, for example, if the lubricant is improperly packaged during manufacturing, if a container sealing fails, or if a neighboring container in a shipment of containers leaks. In addition, as the formulation may be manually applied, residual formulation may be transferred from a user to an exterior surface of a container of lubricant upon manual handling of the container. Thus, it is advantageous to package the inventive formulation in a container that has a slip-resistant exterior surface. Such slip resistant surfaces may be provided, for example, through appropriate selection of container materials or by roughening or otherwise texturing the exterior surface of the container. In general, it is preferred that any exterior surface that may be handled for opening a lubricant container be a slip-resistant surface. For example, when a screw-top bottle is provided as a container for the inventive formulation, it is preferred that either or both of the bottle and the top have an exterior surface that is resistant to slipping. Thus, it is advantageous to provide a container for the inventive formulation, wherein at least about 25% of the exterior surface of the container is resistant to slipping. Preferably, at least about 50% of the exterior surface is slip-resistant. In some instances, substantially the entire exterior surface may be resistant to slipping.

In addition or in the alternative, containers may be equipped with a dispenser-type device (usually mounted on top, as part of a cap assembly) that allows a quantity of the lubricant to be conveniently dispensed when manually operated, such as by depressing a pump mechanism. Such pump-type dispensers are widely used for dispensing creams, ointments, fluidized soaps, or other fluids from such bottles. This allows a desired quantity of the fluid to be placed on the palm or fingers of one hand while the other hand remains dry and clean. In addition, it would allow a genital lubricant to be placed directly onto the penile, vaginal, or vulvar tissue, without getting any of the lubricant onto either hand.

Furthermore, the inventive formulation may be used as lubricant for a barrier contraceptive device. Thus, the formulation may be a condom lubricant that is spread across one or more surfaces of a condom, and that is contained within a sealed watertight package that contains a condom. In other words, the inventive formulation may be pre-packaged with condoms. Such packages may have a slip-resistant exterior surface (such as those described above) as well.

In addition, the inventive formulation may be packaged with or in an applicator. Vaginal applicators have been designed for use with tampons as well with contraceptive devices. Generally, a vaginal insertion device designed for contraceptive purposes serves to insert and place a gel, foam, or similar fluid deep enough inside a vagina so that the fluid coats and blocks the entrance to the uterus. Such devices, which are widely used with contraceptive gels and foams, typically comprise a smooth cylindrical barrel having a distal end and a proximal end. The distal end may be tapered and properly sized for comfortable insertion into a vagina. In addition, a plunger or piston may be slidably located within the barrel. Typically, the plunger or piston is manually forced into the barrel from the proximal end. This sliding action forces fluid out of the barrel through an orifice at the distal end of the barrel.

Two different forms of such insertion devices are commercially available. The first is a disposable single-use form, with a gel-type fluid already loaded inside the chamber, and with the entire article inside a sealed sterile package, for use prior to a single act of intercourse. The second is a reusable device that can be filled and used repeatedly, prior to each act of intercourse, from a container that holds a sufficient quantity of gel or foam for multiple applications. Either type of device is well suited for use with the inventive lubricants as described herein.

It is to be understood that while the invention has been described in conjunction with the preferred specific embodiments thereof, that the foregoing description is intended to illustrate and not limit the scope of the invention. Other aspects, advantages, and modifications within the scope of the invention will be apparent to those skilled in the art to which the invention pertains.

All patents, patent applications, patent publications, and non-patent literature references mentioned herein are incorporated by reference in their entireties.

1. A method for treating a female individual who suffers from or is prone to dyspareunia, comprising administering to the vagina and/or vulvar region of the individual a formulation comprising a therapeutically effective amount of a carrageenan and a pharmaceutically acceptable aqueous carrier.

2. The method of claim 1, wherein the carrageenan is comprised of at least one of lambda carrageenan, kappa carrageenan, and iota carrageenan.

3. The method of claim 2, wherein the formulation is substantially free of any carrageenan other than lambda carrageenan.

4. The method of claim 3, wherein the formulation is substantially free of iota carrageenan.

5. The method of claim 3, wherein the formulation is substantially free of kappa carrageenan.

6. The method of claim 1, wherein the formulation is substantially free of gelled materials.

7. The method of claim 1, wherein the carrageenan is present at a concentration in the range of approximately 1.0 wt. % to approximately 3.0 wt. %.

8. The method of claim 7, wherein the carrageenan is present at a concentration in the range of approximately 1.9 wt. % to approximately 2.1 wt. %.

9. The method of claim 1, wherein the individual exhibits at least one symptom selected from pain and discomfort during sexual intercourse, post-coital vaginal burning, vaginal dryness, vaginal itching, vaginal atrophy, pelvic aching, and urinary discomfort.

10. The method of claim 1, wherein the individual suffers from a sexual dysfunction manifested in part by dyspareunia.

11. The method of claim 10, wherein the sexual dysfunction is an excitement stage sexual dysfunction.

12. The method of claim 1, wherein the formulation has a viscosity of approximately 1,000 to approximately 50,000 centipoise.

13. The method of claim 12, wherein the viscosity is approximately 10,000 to approximately 40,000 centipoise.

14. The method of claim 13, wherein the viscosity is approximately 20,000 to approximately 35,000 centipoise.

15. The method of claim 1, wherein the formulation further comprises a pH-adjusting additive for maintaining the pH of the formulation at an acidic level.

16. The method of claim 15, wherein the means for maintaining the formulation pH at an acidic level is a buffer system.

17. The method of claim 16, wherein the buffer system maintains the formulation at a pH in the range of approximately 3.0 to approximately 5.0.

18. The method of claim 17, wherein the buffer system maintains the formulation at a pH in the range of approximately 3.75 to approximately 4.25.

19. The method of claim 18, wherein the buffer system maintains the formulation at a pH of approximately 4.0.

20. The method of claim 15, wherein the means for maintaining the formulation pH at an acidic level comprises an effective pH-adjusting amount of lactobacillus.

21. The method of claim 1, wherein an acid is added to the formulation no more than about 1 day before administration of the formulation.

22. The method of claim 21, wherein an acid is added to the formulation no more than about 1 hour before administration of the formulation.

23. The method of claim 1, wherein the formulation is free of compounds that compromise the microbial barrier properties of a membrane composed of an elastomeric and/or rubber-based material.

24. The method of claim 1, wherein the formulation is an aqueous solution.

25. The method of claim 1, wherein the formulation is administered using an applicator.

26. The method of claim 1, wherein the formulation further comprises a therapeutically effective amount of a vasoactive agent.

27. The method of claim 26, wherein the vasoactive agent is a vasodilator.

28. The method of claim 27, wherein the vasodilator is selected from the group consisting of prostaglandins, endothelin-derived relaxation factors, smooth muscle relaxants, leukotriene inhibitors, pharmaceutically acceptable salts, esters, analogs, derivatives, prodrugs, active metabolites, and inclusion complexes thereof, and combinations of any of the foregoing.

29. The method of claim 28, wherein the vasodilator is a prostaglandin selected from the group consisting of naturally occurring prostaglandins, synthetic prostaglandins, pharmaceutically acceptable salts, esters, analogs, derivatives, prodrugs, active metabolites, and inclusion complexes thereof, and combinations of any of the foregoing.

30. The method of claim 29, wherein the prostaglandin is selected from the group consisting of PGE₀, PGE₁, PGA₁, PGB₁, PGF_{1α}, 19-hydroxy-PGA₁, 19-hydroxy-PGB₁, PGE₂, PGA₂, PGB₂, 19-hydroxy-PGA₂, 19-hydroxy-PGB₂, PGE₃, PGF_{3α}, PGI₂, and hydrolyzable esters thereof.

31. The method of claim 30, wherein the prostaglandin is prostaglandin E₁.

32. The method of claim 29, wherein the prostaglandin is selected from the group consisting of carboprost tromethamine, dinoprost tromethamine, gemeprost, metenoprost, sulprostone and tiaprost.

33. A pharmaceutical formulation for administration to mucosal tissue, consisting essentially of approximately 1 wt. % to approximately 3 wt. % lambda carrageenan, at least one pH-adjusting additive for maintaining the pH of the formulation at an acidic level, and a pharmaceutically acceptable aqueous carrier suitable for mucosal administration.

34. The formulation of claim 33, wherein the carrier is suitable for vaginal, vulvar, and/or rectal administration.

35. The formulation of claim 33, wherein the formulation is substantially free of gelled materials.

36. The formulation of claim 33, wherein the carrageenan is present at a concentration in the range of approximately 1.9 wt. % to approximately 2.1 wt. %.

37. The formulation of claim 33, wherein the formulation has a viscosity of approximately 1,000 to approximately 50,000 centipoise.

38. The formulation of claim 37, wherein the viscosity is approximately 10,000 to approximately 40,000 centipoise.

39. The formulation of claim 38, wherein the viscosity is approximately 20,000 to approximately 35,000 centipoise.

40. The formulation of claim 33, wherein the pH-adjusting additive for maintaining the formulation pH at an acidic level is a buffer system.

41. The formulation of claim 40, wherein the buffer system maintains the formulation at a pH in the range of approximately 3.0 to approximately 5.0.

42. The formulation of claim 41, wherein the buffer system maintains the formulation at a pH in the range of approximately 3.75 to approximately 4.25.

43. The formulation of claim 42, wherein the buffer system maintains the formulation at a pH of approximately 4.0.

44. The formulation of claim 33, wherein the pH-adjusting additive for maintaining the formulation pH at an acidic level comprises an effective pH-adjusting amount of lactobacillus.

45. The formulation of claim 33, wherein the formulation is free of compounds that compromise the microbial barrier properties of a membrane composed of an elastomeric and/or rubber-based material.

46. The formulation of claim 33, wherein the formulation is an aqueous solution.

47. The formulation of claim 33, wherein the formulation is contained in an applicator.

48. The formulation of claim 47, wherein the applicator is shaped for vaginal insertion.

49. A pharmaceutical formulation for treating dyspareunia, comprising a therapeutically effective amount of a carrageenan, a therapeutically effective amount of a vasoactive agent, and a pharmaceutically acceptable aqueous carrier suitable for vaginal and/or vulvar administration.

50. The formulation of claim 49, wherein the carrageenan is comprised of lambda carrageenan.

51. The formulation of claim 50, wherein the formulation is substantially free of any carrageenan other than lambda carrageenan.

52. The formulation of claim 50, wherein the formulation is substantially free of iota carrageenan.

53. The formulation of claim 50, wherein the formulation is substantially free of kappa carrageenan.

54. The formulation of claim 49, wherein the carrageenan is present at a concentration in the range of approximately 1.0 wt. % to approximately 3.0 wt. %.

55. The formulation of claim 54, wherein the carrageenan is present at a concentration in the range of approximately 1.9 wt. % to approximately 2.1 wt. %.

56. The formulation of claim 49, wherein the formulation has a viscosity of approximately 1,000 to approximately 50,000 centipoise.

57. The formulation of claim 56, wherein the viscosity is approximately 10,000 to approximately 40,000 centipoise.

58. The formulation of claim 57, wherein the viscosity is approximately 20,000 to approximately 35,000 centipoise.

59. The formulation of claim 49, wherein the formulation further comprises a pH-adjusting additive for maintaining the pH of the formulation at an acidic level

60. The formulation of claim 59, wherein the pH-adjusting additive for maintaining the formulation pH at an acidic level is a buffer system.

61. The formulation of claim 60, wherein the buffer system maintains the formulation at a pH in the range of approximately 3.0 to approximately 5.0.

62. The formulation of claim 61, wherein the buffer system maintains the formulation at a pH in the range of approximately 3.75 to approximately 4.25.

63. The formulation of claim 59, wherein the pH-adjusting additive for maintaining the formulation pH at an acidic level comprises an effective pH-adjusting amount of lactobacillus.

64. The formulation of claim 49, wherein the formulation is free of compounds that compromise the microbial barrier properties of a membrane composed of an elastomeric and/or rubber-based material.

65. The formulation of claim 64, wherein the formulation is an aqueous solution.

66. The formulation of claim 49, wherein the vasoactive agent is a vasodilator.

67. The formulation of claim 66, wherein the vasodilator is selected from the group consisting of prostaglandins, endothelin-derived relaxation factors, smooth muscle relaxants, leukotriene inhibitors, pharmaceutically acceptable salts, esters, analogs, derivatives, prodrugs, active metabolites, and inclusion complexes thereof, and combinations of any of the foregoing.

68. The formulation of claim 67, wherein the vasodilator is a prostaglandin selected from the group consisting of naturally occurring prostaglandins, synthetic prostaglandins, pharmaceutically acceptable salts, esters, analogs, derivatives, prodrugs, active metabolites, and inclusion complexes thereof, and combinations of any of the foregoing.

69. The formulation of claim 68, wherein the prostaglandin is selected from the group consisting of PGE₀, PGE₁, PGA₁, PGB₁, PGF_{1α}, 19-hydroxy-PGA₁, 19-hydroxy-PGB₁, PGE₂, PGA₂, PGB₂, 19-hydroxy-PGA₂, 19-hydroxy-PGB₂, PGE₃, PGF_{3α}, PGI₂, and hydrolyzable esters thereof.

70. The formulation of claim 69, wherein the prostaglandin is prostaglandin E₁.

71. The formulation of claim 70, wherein the prostaglandin is selected from the group consisting of carboprost tromethamine, dinoprost tromethamine, gemeprost, metenoprost, sulprostone and tiaprost.

72. The formulation of claim 49, wherein the formulation is contained in an applicator.

73. The formulation of claim 72, wherein the applicator is shaped for vaginal insertion.

74. A method for enhancing vaginal lubrication, comprising administering to the vagina and/or vulvar region of a healthy female individual a formulation consisting essentially of a therapeutically effective amount of a carrageenan, a pharmaceutically acceptable aqueous carrier, an optional pH-adjusting additive for maintaining the pH of the formulation at an acidic level and an optional vasoactive agent.

75. A method for enhancing vaginal lubrication, comprising administering to the vagina and/or vulvar region of a female individual a formulation consisting essentially of approximately 1 wt. % to approximately 3 wt. % lambda carrageenan, a pharmaceutically acceptable aqueous carrier, an optional pH-adjusting additive for maintaining the pH of the formulation at an acidic level and an optional vasoactive agent.

76. A packaged kit for an individual to use in the administration of a personal lubricant, comprising the pharmaceutical formulation of claim 33, a container housing the pharmaceutical formulation during storage and prior to administration, and instructions for a patient to self-administer the formulation.

77. A packaged kit for an individual to use in the administration of a personal lubricant, comprising the pharmaceutical formulation of claim 49, a container housing the pharmaceutical formulation during storage and prior to administration, and instructions for a patient to self-administer the formulation.
